(19) United States
(12) Patent Application Publication
(10) Pub. No.: US 2025/0050073 A1
(43) Pub. Date: Feb. 13, 2025

(54) Clip on Torque Device for Endovascular Procedures

(71) Applicant: Wisconsin Alumni Research Foundation, Madison, WI (US)

(72) Inventors: Dai Yamanouchi, Madison, WI (US); Isabelle Gundrum, Northbrook, IL (US); William Hayes, Eau Claire, WI (US); Anna Ankerstjerne, Eagan, MN (US); Eric Sagen, Madison, WI (US); Jakob Knauss, Madison, WI (US)

(21) Appl. No.: 18/231,997

(22) Filed: Aug. 9, 2023

(51) Int. Cl. A61M 25/09

(52) U.S. Cl. CPC A61M 25/09041

(57) ABSTRACT

A torque device employs a cylindrical housing surrounding a guidewire to provide concentric loading of the guidewire along a central axis of the housing attachable at any point along a length of the guidewire. Improved torsion of the guidewire is obtained by clamping the guidewire so that it runs along the center axis of the housing offering intuitive rotational handling (torque) of the guidewire along the torque axis.

### STATEMENT REGARDING FEDERALLY SPONSORED RESEARCH OR DEVELOPMENT

### CROSS REFERENCE TO RELATED APPLICATION

### BACKGROUND OF THE INVENTION

The present invention relates to a medical wire manipulation device for controlling the linear and rotational (torque) motion of a wire and, in particular, to an improved torque device for making such controlling movements along the length of a guidewire during a minimally invasive procedure.

Spanning a broad range of medical disciplines including cardiology, neurology, urology, interventional radiology, and endovascular surgery, minimally invasive procedures have been on the rise and can range from treatment of narrowed arteries and veins, to insertion of drains and emergency hemorrhage control. Additionally, minimally invasive techniques can be used for diagnostic imaging, and interventional procedures or intervention focuses on performing minimally invasive, image-guided procedures throughout the body. The increasing number of these minimally invasive procedures indicates that small improvements in these procedures can have big impacts on improved surgical workflow and future patient outcomes.

In one non-limiting example, intervention for the treatment of cardiovascular disease has been gravitating towards minimally invasive methods. Endovascular procedures are increasingly common for minimally invasive treatment of conditions associated with cardiovascular disease, such as peripheral artery disease, deep vein thrombosis, coronary artery disease, and aneurysms. For example, there has been a significant shift in treating abdominal aortic aneurysms using minimally invasive endovascular procedures, with a 76% decrease in traditional open surgery. This increase in endovascular interventions, compared to open surgery, has resulted in lower associated costs, shorter recovery times, and reduced negative impact on the patient.

For many minimally invasive techniques, time-critical navigation of tortuous vasculature is required, demanding intricate manipulation of a guidewire. Torque devices are the ubiquitous method for providing fine-precision control of the guidewire. Torque devices help physicians advance, rotate, and grip the guidewire that is used to guide catheters to the desired location within the vascular system. Commonly used torque devices operate using a pin vise mechanism. This mechanism screws concentrically around the distal end of the guidewire and therefore must be loaded 180-300 cm from the incision. Overtightening may cause damage to the guidewire. Moreover, this cumbersome maneuver requires assistance from a second individual.

Any procedure change or additional tool available to the physician that aids in decreasing procedure times is an opportunity to improve patient outcomes. Thus, there is an opportunity to improve torque devices to increase usability for the physician and further reduce procedure times during minimally invasive procedures.

### SUMMARY OF THE INVENTION

The current market leaves a gap that can be filled by a torque device that is attachable at any position along the length of the guidewire, not just the distal end, with a single hand and without assistance. This increases usability and further reduces procedure times during minimally invasive procedures such as endovascular interventions.

The present invention provides a torque device that employs a cylindrical housing surrounding a guidewire to provide concentric loading of the guidewire along a central axis of the housing and attachable at any point along a length of the guidewire. Improved torsion of the guidewire is obtained by clamping the guidewire so that it runs along the center axis of the housing offering intuitive rotational handling (torque) of the guidewire along the torque axis. The housing increases the grip diameter of the guidewire improving the handling and “torqueability” of the guidewire or the ability to apply rotational force at the distal end or along the guidewire and have that force transmitted efficiently to achieve proper control at the proximal (insertion) end. This is an improvement over torque devices that are attached at the distal end only or fail to provide concentric clamping of the guidewire.

Further, the present invention provides a spring loaded torque device that clamps the guidewire along the central axis of the housing without adding unnecessary side bulk to the device or presenting an asymmetry to the device providing a less intuitive handling.

Further, the present invention provides a sufficient clamping force on the guidewire regardless of wire size that minimizes slipping while mitigating damage to the guidewire.

In one embodiment, the invention provides a device for manipulating a guidewire for surgery, comprising a housing adapted to receive a wire, insertable into a patient at an incision site, the housing having a slot receiving a center portion of the wire between a proximal end and distal end of the wire wherein the wire is positionable to extend on a central axis of the housing; and the housing supporting a clamp assembly having a pincher and a biasing member configured to apply a biasing force on the pincher to bias the pincher to contact the wire at an at least one discrete pinch area to prevent movement of the wire with respect to the pincher.

It is thus a feature of at least one embodiment of the invention to create a concentric housing that provides a greater diameter grip section of the guidewire increasing torqueability.

The housing may be cylindrical in shape extending along the central axis of the housing.

It is thus a feature of at least one embodiment of the invention to facilitate rotation (torque) of the housing about the central axis by using a rounded cylindrical device that can be easily rotated between two fingers.

The housing may have at least two opposed flattened outer surfaces.

It is thus a feature of at least one embodiment of the invention to assist with gripping the cylindrical housing when the housing is slippery without adversely affecting the rotatability of the cylindrical housing.

The housing may be substantially concentric about an axis of the wire.

It is thus a feature of at least one embodiment of the invention to create intuitive rotation of the guidewire which corresponds to the rotation of the housing on the same axis where finely tuned torque precision is desired.

The slot may extend from a sidewall of the housing to the central axis. The slot has a height that is between 1 mm to 3 mm or between 1.9304 mm and 2.8956 mm.

It is thus a feature of at least one embodiment of the invention to allow the housing to be installed along the length of the guidewire so that the guidewire can be handled closer to the incision site/proximal end rather than at the distal end thus providing better hand control.

A groove may be formed on an inner wall of the slot receiving the wire substantially on the center axis. The groove may be V-shaped.

It is thus a feature of at least one embodiment of the invention to align the wire on the central axis regardless of the diameter of the wire and to position the wire along the pinch point locations where pinching contact can be confidently made.

At least one discrete pinch area may be discrete points on the wire. The pincher may include at least one outwardly extending pinching tooth. The pincher may include two pinching teeth.

It is thus a feature of at least one embodiment of the invention to minimize damage to the wire, caused by applying excessive force, and by limiting the force contact areas to discrete pinch points.

The biasing member may include at least one spring.

It is thus a feature of at least one embodiment of the invention to offload the desired pinching force to spring loaded clamps where the clamping force can be modified by changing the number of springs and/or the spring constant of the selected springs.

The clamp assembly may comprise a jaw supporting the pincher and movable against the biasing force of the biasing member. The jaw may comprise an upper arm opposite a lower arm. At least one of the upper arm and lower arm support the pincher and the other of the upper arm and lower arm support the biasing member.

It is thus a feature of at least one embodiment of the invention to reduce the number of separate parts by incorporating the push button and pincher into a single movable clamp assembly.

A button may be pressable by a human finger to move the pincher against the biasing force of the biasing member.

It is thus a feature of at least one embodiment of the invention to allow the housing to be installed on the guidewire at any location along the guidewire through one handed, one finger push button operation without the assistance of a second individual.

One embodiment of the present invention provides a method for manipulating a guidewire having a central portion between a proximal end and distal end of the wire for surgery comprising inserting the proximal end of the guidewire into a patient at an incision site, inserting the central portion of the guidewire into a slot of a housing wherein the guidewire is positionable to extend on a center axis of the housing; and clamping a clamp assembly to contact the guidewire at an at least one discrete pinch area.

The clamp assembly may apply a biasing force to contact the guidewire at the at least one discrete pinch area.

The method may further comprise pressing a button to apply an opposite force against the biasing force of the clamp assembly to release the guidewire at the at least one discrete pinch area.

The method may further comprise inserting the central portion of the guidewire into a groove of the slot of the housing wherein the groove extends on the center axis of the housing.

These particular objects and advantages may apply to only some embodiments falling within the claims and thus do not define the scope of the invention.

### BRIEF DESCRIPTION OF THE DRAWINGS

FIG. 1 is a perspective view of the torque device held by a hand of a physician in accordance with one embodiment of the present invention and may be positioned on a guidewire between the proximal end (inserted into a patient) and distal end to hold a guidewire on a central axis of a housing of the torque device;

FIG. 2 is an exploded view of the torque device of FIG. 1 showing the relative attachment of the left housing part and right housing part by corresponding pegs and holes to form an internal cavity for movably supporting a clamping device positioned therebetween;

FIG. 3 is a perspective view of the torque device of FIGS. 1 and 2 shown in partial phantom showing a push button protruding from the bottom of the housing and the clamping device spring biased in a “clamping position” to clamp the guidewire on the central axis;

FIG. 4 is a cross sectional view of the torque device of FIG. 3 showing the clamping device spring biased in the “clamping position” to clamp the guidewire on the central axis and the push button protruding from the bottom of the housing; and

FIG. 5 is a cross sectional view of the torque device similar to FIG. 4 showing the clamping device in a “non-clamping position” to allow the guidewire to be inserted along the central axis, or release the guidewire, when the push button is pressed into the housing.

### DETAILED DESCRIPTION OF THE PREFERRED EMBODIMENT

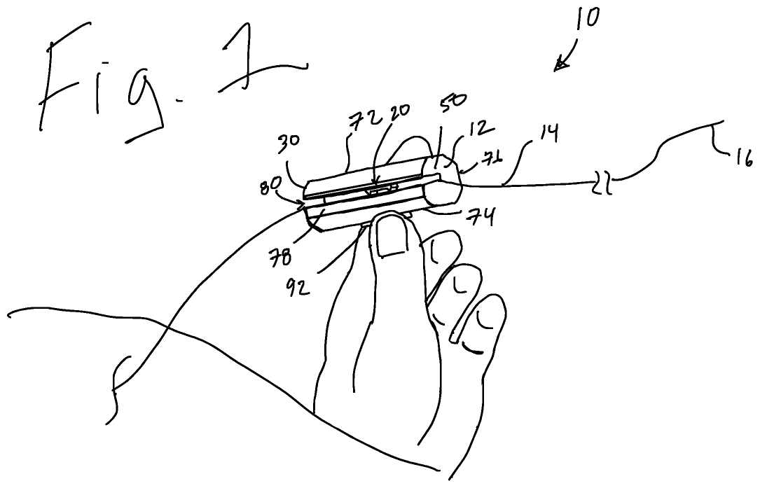

Referring now to FIG. 1, a torque device 10 constructed according to one embodiment of the present invention may provide a torque housing 12. The torque housing 12 is sized and shaped to receive a guidewire 14 therein, the guidewire 14 being further inserted into an opening or passage of a patient during a minimally invasive procedure. In use, a catheter may be guided over the guidewire 14 to assist with the minimally invasive interventional procedure as understood in the art. In one non-limiting example, the guidewire 14 is guided into a blood vessel to perform vascular and endovascular surgery, including complex stenting procedures, blood clot removal, and bypass surgery.

The guidewire 14 has a proximal end (not shown) inserted into the patient's body and an opposite distal end 16 with a length therebetween supporting the torque device 10 held and maneuvered by the physician's hand to advance and torque the guidewire 14. The guidewire 14 may have an overall length ranging from 50 cm to 500 cm in length and have diameter sizes ranging from 0.3048 mm to 0.9652 mm (0.012 to 0.018 inches). The guidewire 14 has varied tensile strengths (e.g., at least 5 Newton (N)) and may be solid, braided, or coiled in construction. The guidewire 14 may include surface coatings that may increase lubricity and trackability, or assist with attracting or repelling water. Examples of guidewire coatings known in the art include polyvinylpyrrolidone (PVP), polytetrafluoroethylene (PTFE), polyacrylamides, hyaluronic acid, water-trapping hydrogels, and the like.

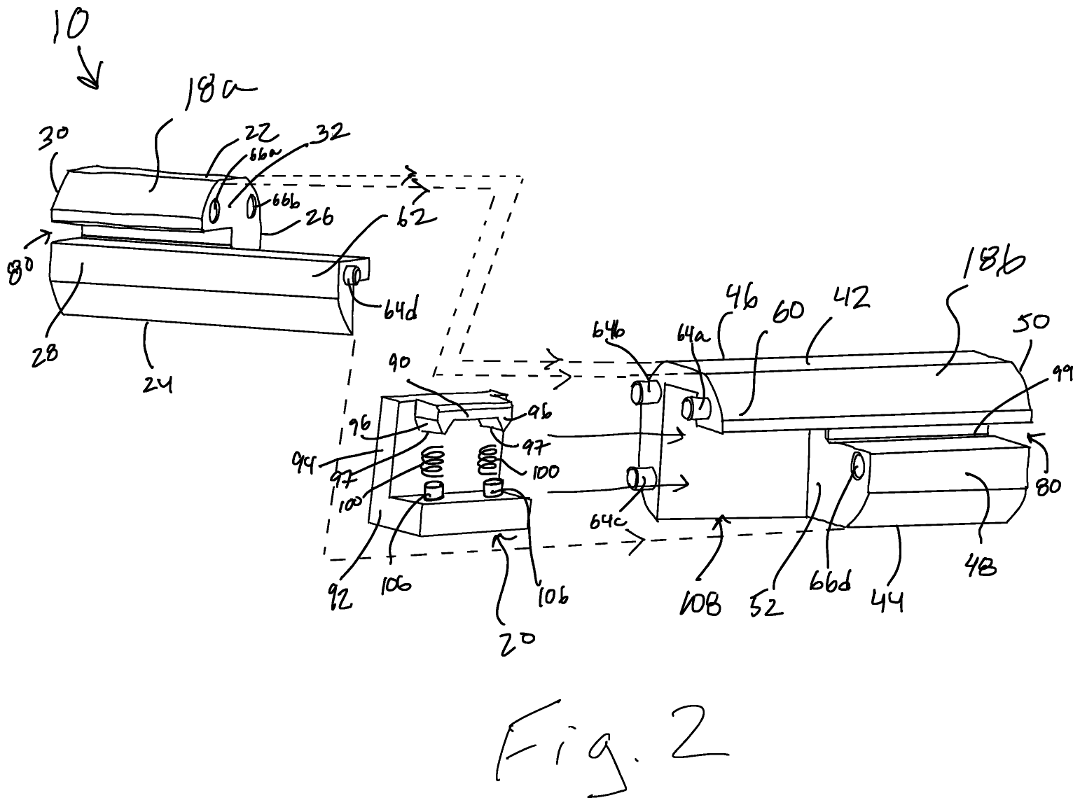

Referring also to FIG. 2, the torque housing 12 may be defined by a cylindrical shell formed by joining two separate cylindrical connectable housing parts 18a, 18b along corresponding connectable side walls to produce a singular torque housing 12 further supporting a clamp assembly 20. The connectable housing parts 18a, 18b may be manufactured of injection molded plastics such as polyethylene, polypropylene, polystyrene, polycarbonate, polyetheretherketone (PEEK), and the like.

A first housing part 18a includes an upper wall 22 opposite a lower wall 24 and flanking a rear wall 26 opposite a front wall 28. An outer left sidewall 30 extends opposite a mating right sidewall 32, with the mating right sidewall 32 couplable to the second housing part 18b. The second housing part 18b includes an upper wall 42 opposite a lower wall 44 and flanking a rear wall 46 opposite a front wall 48. An outer right sidewall 50 extends opposite a mating left sidewall 52, with the mating left sidewall 52 couplable to the first housing part 18a.

The mating right sidewall 32 of the first housing part 18a and the mating left sidewall 52 of the second housing part 18b interlock to provide an overlap of an upper projection 60 of the mating left sidewall 52 of the second housing part 18b over a lower projection 62 of the mating right sidewall 32 of the first housing part 18a. The upper projection 60 joins the mating right sidewall 32 and the lower projection 62 joins the mating left sidewall 52. When joined, the connectable housing parts 18a, 18b leave hollow spaces therebetween that, when joined, form a cavity which permits the clamp assembly 20 to be supported therein, as further described below.

The upper projection 60 and the mating right sidewall 32, and the lower projection 62 and the mating left sidewall 52, respectively, may be joined by outwardly extending pegs 64 and corresponding holes 66. The interlocking pegs 64 and holes 66 may be cylindrical pegs secured by an interference fit into corresponding cylindrical holes, and optionally, may be further secured by using an adhesive. For example, the pegs 64 may have a diameter of about 1.36 mm and the corresponding holes 66 may have a slightly larger diameter of about 1.72 mm. The corresponding pegs 64 and holes 66 may have lengths and depths, respectively, of about 1 mm.

In one embodiment, the outwardly extending pegs 64 may extend from the upper projection 60 and the lower projection 62 and the corresponding holes 66 may be formed in the mating right sidewall 32 and the mating left sidewall 52, however, it is understood that the location of the outwardly extending pegs 64 and corresponding holes 66 may be reversed or may be positioned at any location on the mating right sidewall 32 and the mating left sidewall 52.

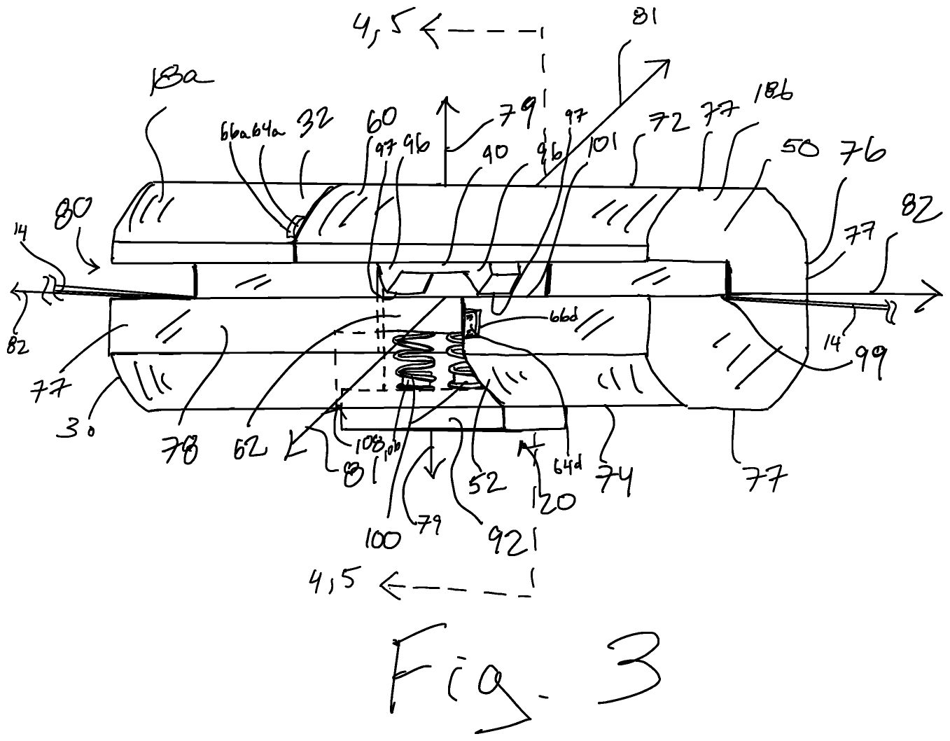

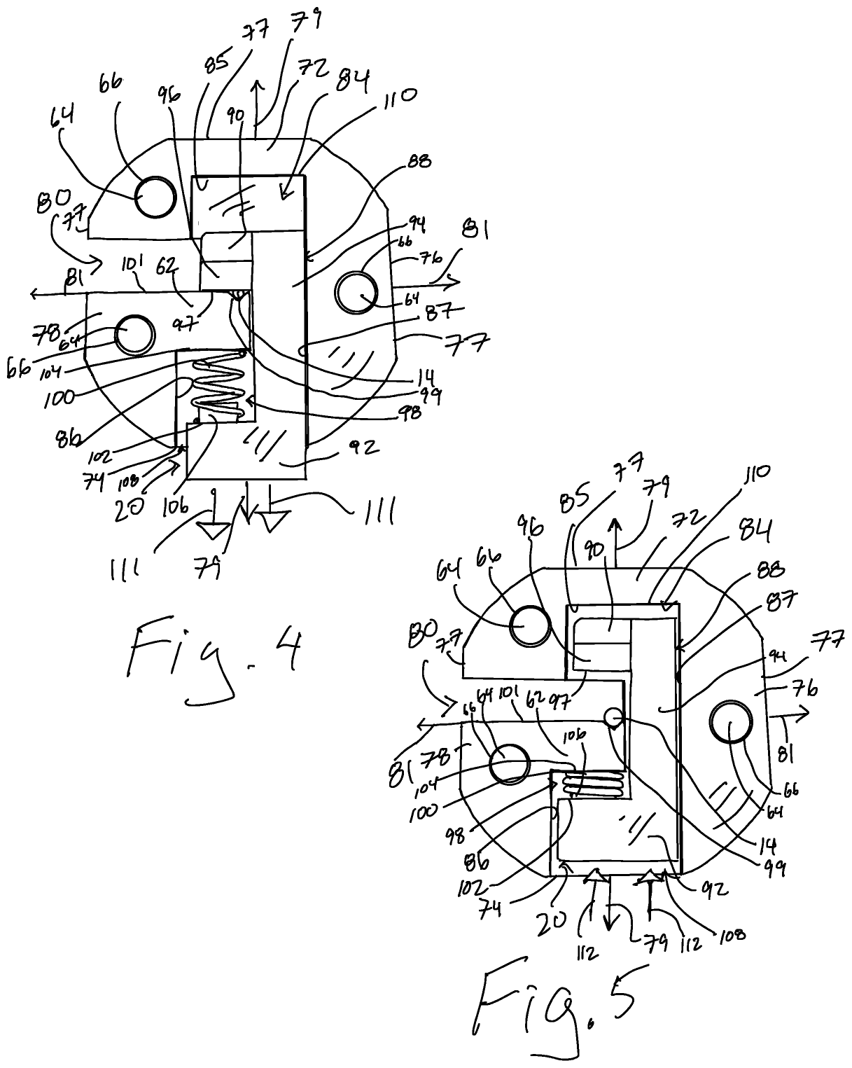

The upper projection 60 and the mating right sidewall 32, and the lower projection 62 and the mating left sidewall 52, may include one or more corresponding pegs 64 and holes 66. In one embodiment, as seen in FIGS. 2 and 3, the upper projection 60 may support a first peg 64a, a second peg 64b, and third peg 64c insertable into a first hole 66a, a second hole 66b, and third hole (hidden from view), respectively, of the mating right sidewall 32, and the lower projection 62 supports a fourth peg 64d insertable into a third hole 66d of the mating left sidewall 52. It is understood that the number and configuration of the outwardly extending pegs 64 and corresponding holes 66 may vary, e.g., as seen in the alternative embodiment seen in FIGS. 4 and 5 showing three pegs 64 and three holes 66, to secure the connectable housing parts 18a, 18b together.

Although outwardly extending pegs 64 and corresponding holes 66 are described herein, it is understood that the mating right sidewall 32 and the mating left sidewall 52, may be joined by other attachment means known in the art such as using snap fits, mechanical fastening, welding, adhesive and solvent bonding, UV bonding, and the like. It is also understood that the torque housing 12 may be formed as a unitary structure instead of joinable parts without deviating from the spirit of the invention.

Referring now to FIG. 3, upon joining the first housing part 18a and second housing part 18b, the torque housing 12 forms a generally cylindrical shell with a central axis 82 extending along a length of the housing 12 and having a housing upper wall 72 (formed by the joining of the upper wall 22 and upper wall 42) opposite a housing lower wall 74 (formed by the joining of the lower wall 24 and lower wall 44), and flanking a housing rear wall 76 (formed by the joining of the rear wall 26 and rear wall 46) opposite a housing front wall 78 (formed by the joining of the front wall 28 and front wall 48). The torque housing 12 has a height, extending along a vertical axis 79 and between the housing upper wall 72 and the housing lower wall 74 that is between 10 to 15 mm and about 11.4 mm. The torque housing 12 has a length, extending along the central axis 82 of the torque housing 12 and between the outer left sidewall 30 and the outer right sidewall 50, that is between 30 to 50 mm and about 39 mm. The torque housing 12 has a depth, extending along a horizontal axis 81 and between the housing front wall 78 and housing rear wall 76, that is between 10 to 15 mm and about 11.4 mm. The torque housing 12 may have dimensions that are less than 50 mm by 15 mm by 15 mm and less than 40 mm by 12 mm by 12 mm.

The housing upper wall 72, housing lower wall 74, housing rear wall 76, and housing front wall 78 may be slightly flattened on its outer surfaces 77 to provide opposed gripping surfaces to receive the physician's opposed fingers and that minimize accidental slipping that may occur, for example, when the physician's fingers are slippery when wet.

Referring also to FIGS. 4 and 5, the torque housing 12 may include a guidewire slot 80 extending along the central axis 82 allowing the guidewire 14 to be inserted into an interior of the torque housing 12 along the central axis 82. The guidewire slot 80 may be a hollow channel formed in the housing front wall 78 and extending inwardly from the housing front wall 78 to the central axis 82 along a horizontal axis 81 terminating on the central axis 82.

The guidewire slot 80 may be a rectangular channel having a height measured along the vertical axis 79 between 1.0 mm to 3.0 mm; and at least 1.0 mm and at least 1.5 mm and at least 1.6 mm and at least 1.7 mm and at least 1.8 mm and at least 1.9 mm; and less than 3.0 mm and less than 2.9 mm and less than 2.8 mm and less than 2.7 mm and less than 2.6 mm and less than 2.5 mm, and corresponding to about or at least twice the diameter of the largest anticipated guidewire 14 diameter and less than three times the diameter of the largest anticipated guidewire 14 diameter (the largest anticipated guidewire diameter is about 0.9652 mm) or between about 1.9304 mm and 2.8956 mm. The height of the guidewire slot 80 permits easy insertion of the guidewire 14 but is not too wide to diminish the stabilizing force on the peripheral areas of the guidewire 14 surrounding the pinch points. The guidewire slot 80 may have a length measured along the axis 82 between 30 to 50 mm and about 39 mm and corresponding to the length of the torque housing 12. The guidewire slot 80 may have a depth measured along the horizontal axis 81 between 5 to 8 mm and about 6.11 mm and corresponding to about half the depth of the torque housing 12 and extending to the central axis 82.

The guidewire slot 80 extends to the central axis 82 of the torque housing 12 thus allowing the guidewire 14 to be placed on and along the central axis 82. The bottom wall 101 of the guidewire slot 80 may include an alignment groove 99 assisting with the positioning of the guidewire 14 within the guidewire slot 80 on and along the central axis 82. The alignment groove 99 may have a V-shaped (like a V-block), round, or rectangular cut out or groove (when viewed in cross section as seen in FIGS. 4 and 5) extending along the central axis 82 and receiving a bottom side of the guidewire 14 but permitting the top side of the guidewire 14 to project upwardly from the alignment groove 99 (even for the smallest anticipated guidewire diameter of 0.3048 mm). In this respect, the alignment groove 99 does not eliminate or reduce contact of the top side of the guidewire 14 with the clamp assembly 20 which is necessary to contact the guidewire 14 to prevent slipping (when linear and torque force is applied) as further discussed below.

The mating right sidewall 32 of the first housing part 18a and the mating left sidewall 52 of the second housing part 18b may include hollow spaces therebetween that join to form a clamp cavity 84 (see FIGS. 4 and 5 in cross section) between the first housing part 18a and second housing part 18b. The clamp cavity 84 may be sized and shaped to receive the movable clamp assembly 20 and provide restricted linear vertical movement, as further described below.

The clamp assembly 20 includes a C-shaped jaw 88 having an upper horizontal arm 90 extending along the housing upper wall 72 and a lower horizontal arm 92 extending along the housing lower wall 74. The upper horizontal arm 90 and lower horizontal arm 92 are joined by a vertical bridge 94 extending along the housing rear wall 76.

The upper horizontal arm 90 has a length measured along the central axis 82 between 6 to 10 mm and about 8.10 mm and a height measured along the vertical axis between 1 to 3 mm and about 1.15 mm and a depth measured forwardly from the vertical bridge 94 between 1.5 to 3 mm and about 2.2 mm. The lower horizontal arm 92 has a length measured along the central axis 82 between 10 to 12 mm and about 11.7 mm and a height measured along the vertical axis between 1.5 to 3 mm and about 2.3 mm and a depth measured forwardly from the vertical bridge 94 between 1.5 to 4 mm and about 3.2 mm. The upper horizontal arm 90 may have a slightly shorter height, shorter depth, and shorter length than the lower horizontal arm 92. The vertical bridge 94 has a length measured along the central axis 82 between 10 to 15 mm and about 11.7 mm and a height measured along the vertical axis 79 between 8 to 10 mm and about 9.15 mm and a depth measured along the horizontal axis 81 between 1 to 3 mm and about 1.8 mm.

The clamp cavity 84 is C-shaped with an upper horizontal cavity 85, a lower horizontal cavity 86, and a vertical cavity 87 receiving the upper horizontal arm 90, lower horizontal arm 92 and vertical bridge 94, respectively, of the C-shaped jaw 88. The C-shaped jaw 88 is restrained from substantial movement along the central axis 82 by the mating right sidewall 32 and the mating left sidewall 52 (as best seen in FIG. 3). The C-shaped jaw 88 is restrained from substantial movement along the horizontal axis 81 by the housing front wall 78 and the housing rear wall 76 (as best seen in FIGS. 4 and 5). The C-shaped jaw 88 is movable along the vertical axis 79 but is restricted along the vertical axis 79 by the housing upper wall 72 on a top end and by the rightward extending lower projection 62 (separating the upper horizontal cavity 85 and lower horizontal cavity 86) engaging the upper horizontal arm 90 on a lower end and preventing the upper horizontal arm 90 from moving further downward (as best seen in FIGS. 4 and 5).

Generally, the clamp cavity 84 is sized and shaped to support the C-shaped jaw 88 and a biasing member 98 to permit the C-shaped jaw 88 to move between (1) a “clamping position” where the upper horizontal arm 90 is biased downward against a bottom wall 101 of the guidewire slot 80 and the lower horizontal arm 92 extends outwardly from a rectangular opening 108 of the housing lower wall 74 per FIGS. 4 and (2) a “non-clamping position” or “released position” where the upper horizontal arm 90 is raised above the bottom wall 101 and the lower horizontal arm 92 is pressed upwardly into the lower projection 62 of the housing lower wall 74 against the spring bias per FIG. 5.

The upper horizontal arm 90 supports a pincher being a pair of downwardly extending teeth 96 for gripping the guidewire 14 extending within the alignment groove 99 of the guidewire slot 80 in the “clamping position.” The downwardly extending teeth 96 may be a pair of triangular prisms with rectangular bases of the triangular prisms attached to the upper horizontal arm 90 with vertexes of the triangular prisms extending downwardly toward the bottom wall 101 of the guidewire slot 80 and forming a clamp edge 97 extending along the horizontal axis 81 and across (perpendicular to) the alignment groove 99. The alignment groove 99 ensures that the clamp edges 97 of the downwardly extending teeth 96 will properly engage a top side of the guidewire 14 by maintaining the position of the guidewire 14 on and along the central axis 82.

The bases of the triangular prisms may have a length measured along the central axis 82 between 1.5 to 3 mm and about 1.76 mm and spaced apart by a gap between 3 to 6 mm and about 4.74 mm, and a depth measured along the horizontal axis 81 between 1.5 to 3 mm and about 2.2 mm. Although the clamp edges 97 taper to tips, the clamp edges 97 may be slightly curved or rounded at the tips to increase the surface area contact on the guidewire 14 but minimizing damage to the guidewire 14.

The clamp edges 97 of the downwardly extending teeth 96 will contact the guidewire 14 at two discrete contact areas or “pinch points” to hold the guidewire 14 against the bottom wall 101 of the guidewire slot 80. It is understood that the number of downwardly extending teeth 96 and thus pinch points may be varied, for example, one, two or more pinch points. The pinch points minimize damage to the guidewire 14 by providing a greater downward point load on the wire at a small number of discrete points. However, it is understood that the downwardly extending teeth 96 may alternatively provide greater contact areas that are extended lengths of the guidewire 14 to increase the friction force on the guidewire 14. In this respect, the length of the clamp edges 97 of the downwardly extending teeth 96 may be increased to increase the area of contact.

It is understood that the surface contact at the discrete contact areas between the guidewire 14 with the clamp edges 97 creates a friction force that will minimize slipping (when linear and torque force is applied) of the guidewire 14 with respect to the clamp edges 97. The friction force is created through the biasing member 98 applying a force on the guidewire 14, as further described below. Desirably, the clamped guidewire 14 can withstand an average tensile force (linear force) with a maximum value of 5 N for guidewires less than 0.762 mm diameter and 10 N for guidewires greater than 0.762 mm diameter without slipping. Desirably, the clamped guidewire 14 can withstand a torque force which avoids slipping during normal use.

Although it is contemplated that the C-shaped jaw 88 may be manufactured of an injection molded plastic similar to the torque housing 12, the pair of downwardly extending teeth 96 could also be manufactured of a different material to assist with the clamping of the guidewire 14 such as a solid rubber or silicone which may produce an increased “flattened” contact area or may be roughened to increase the friction force on the guidewire 14.

The C-shaped jaw 88 is biased to the “clamping position” which results in the downwardly extending teeth 96 clamping down on the guidewire 14 when an opposing force is not applied. A biasing force of the C-shaped jaw 88 is created by the biasing member 98 which may be springs, spring clips, spring steel, and the like. In one embodiment, the biasing member 98 may be a pair of springs 100 positioned between an upper surface 102 of the lower horizontal arm 92 and a lower surface 104 of the lower projection 62. The pair of springs 100 may be spaced apart on the lower horizontal arm 92 to provide an equal distribution of spring force on the lower horizontal arm 92.

The pair of springs 100 may be held in place by a pair of pegs 106 on at least one of the upper surface 102 of the lower horizontal arm 92 and the lower surface 104 of the lower projection 62 receiving the springs 100 thereon. The pair of pegs 106 may have a length of about 0.58 mm, and a diameter of about 1.20 mm, to support a length and diameter of the pair of springs 100 installed thereon to maintain a fixed position of the springs 100 along the length of the pegs 106 but allowing the springs to extend and compress.

In one embodiment, a first pair of pegs 106 may extend downwardly from the lower surface 104 of the lower projection 62 and/or a second pair of pegs 106 may extend upwardly from the upper surface 102 of the lower horizontal arm 92, as shown, with the pair of springs 100 extending around and supported by the pairs of pegs 106. It is understood that one or both of the lower surface 104 of the lower projection 62 and the upper surface 102 of the lower horizontal arm 92, respectively, may support pegs 106. It is also understood that the pair of springs 100 may be secured between the upper surface 102 of the lower horizontal arm 92 and the lower surface 104 of the lower projection 62 by other securing means known in the art such as spring stops, spring retainers, tape or zip ties, and the like.

Referring to FIG. 4, when the pair of springs 100 is relaxed, the springs push the lower horizontal arm 92 downward along direction arrows 111 away from the lower projection 62, thus moving the upper horizontal arm 90 downward against the bottom wall 101 to apply a pinching force on the guidewire 14. The pair of springs 100 may be selected to provide a spring constant (k) with enough stiffness to apply enough force to retain the guidewire 14 within the alignment groove 99 without sliding but not too stiff to exert excessive force causing damage to the guidewire 14. Further, the spring constant (k) is chosen to allow the physician to press against the pair of spring 100 with a single finger away from the “clamping position,” as further discussed below. In one embodiment, the spring constant (k) is 1225.9 N/m to 5253.8 N/m (7 pounds per inch to 30 pounds per inch) and approximately 15 pounds per inch (2626.9 N/m).

When the lower horizontal arm 92 is spring biased to the “clamping position,” the lower arm 92 extends downwardly and outwardly from the housing lower wall 74 along direction arrows 111 through a rectangular opening 108. The lower horizontal arm 92 may extend outwardly approximately 1 to 2 mm from the housing lower wall 74 through the rectangular opening 108.

Referring now to FIG. 5, the lower horizontal arm 92 is accessible through the rectangular opening 108 in the housing lower wall 74 acting as a push button which allows the physician to finger press (without tools) the lower horizontal arm 92 upward along vertical axis 79 into the housing lower wall 74 to the “non-clamping position” or “released position” against the spring bias of the pair of springs 100 and thereby raising the upper horizontal arm 90 away from the bottom wall 101 releasing the downwardly extending teeth 96 from gripping or pinching the guidewire 14 or allowing a guidewire 14 to be inserted into the alignment groove 99. The lower horizontal arm 92 may be pressed along direction arrows 112 into the housing lower wall 74, for example, pressed approximately 2 to 4 mm upward along vertical axis 79 until the upper horizontal arm 90 abuts the upper wall 110 of the upper horizontal cavity 85.

It is understood that the lower horizontal arm 92 acts as a push button that may be easily pressed along direction arrows 112 by the physician with one finger, for example, the torque housing 12 held between the thumb and index finger and the lower horizontal arm 92 pressed along direction arrows 112 by either the thumb or index finger. The button formed by the lower horizontal arm 92 may be sized approximately 11.7 mm by 5 mm which is approximately the same size as or slightly larger than the pad of a human finger thus permitting the pressure of the finger to evenly apply force onto the button. In this way, finger force is equally applied to the button so that both springs of the pair of springs 100 are compressed to avoid button tilt or slant and to maintain proper balance between springs.

Referring again to FIG. 1, in operation, in a first step, during a minimally invasive surgical procedure the torque device 10 is held by the physician, for example, between their thumb and index finger. The physician may hold the guidewire 14 with their other hand to prepare for loading the torque device 10 onto the guidewire 14.

Referring to FIG. 5, in a second step, the physician uses their finger to press the lower horizontal arm 92 upward into housing lower wall 74 to move the upper horizontal arm 90 of the C-shaped jaw 88 upward away from the bottom wall 101 to the “non-clamping position” or “released position.” The clearance between the downwardly extending teeth 96 and the bottom wall 101 permits the guidewire 14 to be inserted therebetween by the physician.

In a third step, the physician slides a short length of the guidewire 14 (at any position along the length of the guidewire 14) into the guidewire slot 80 so that the guidewire 14 is positioned within the alignment groove 99 of the guidewire slot 80. In one embodiment, the guidewire 14 may be inserted into the guidewire slot 80 at a position that is about 180 to 300 cm from the incision.

Referring now to FIG. 4, in a fourth step, the physician releases the lower horizontal arm 92 to allow the C-shaped jaw 88 to move from the “non-clamping position” or “released position” to the “clamping position.” Upon release of the lower horizontal arm 92, the C-shaped jaw 88 is spring biased to clamp down on the guidewire 14 at the discrete contact areas or pinch points formed by the pair of downwardly extending teeth 96 to retain the guidewire 14 within the alignment groove 99 and on and along the central axis 82.

Referring again to FIG. 1, in a fifth step, the physician may move the torque housing 12 to manipulate the linear and rotational (torque) motion of the guidewire 14 with respect to the incision of the patient. The torque housing 12 provides intuitive handling by maintaining a concentric housing around the central axis 82 which increases a diameter of the guidewire 14 but leaves the central axis 82 of the torque housing 12 the same as an axis of the guidewire 14. The cylindrical housing is easy to handle and rotate between the fingers without unwanted protrusions or asymmetry to the device. Further, the strong clamping of the guidewire 14 provided by the biasing member 98 minimizes unwanted slipping during maneuvering while still being usable with a range of guidewire 14 diameters.

Referring again to FIG. 5, in a sixth step, the physician may remove the torque housing 12 from the guidewire 14 by pressing the lower horizontal arm 92 into the housing lower wall 74 to move the upper horizontal arm 90 of the C-shaped jaw 88 upward away from the bottom wall 101 to the “non-clamping position” or “released position.”

In a final step, the physician slides the released guidewire 14 out of the guidewire slot 80 to remove the guidewire 14 from the torque housing 12. The physician may desire to move the torque housing 12 to a different location along the guidewire 14 or may be ready to remove the guidewire 14 after use.

In certain embodiments, the torque housing 12 may be of various colors or include other visual identifiers to allow the physician to use the color of the torque device 10 as a visual indicator for identifying different guidewires 14, e.g., differently sized guidewires 14, used during surgery.

Further, the position of the torque housing 12 on the guidewire 14 may be used as a location marker to assist with visualizing the length of guidewire 14 inserted into the incision of the patient during the surgery.

In a non-limiting example, the torque device 10 may be used during endovascular surgery and open vascular surgery for conditions that affect the blood vessels and heart. In traditional open vascular surgery, incisions are made at the affected area, while endovascular surgery utilizes a minimally invasive approach. To perform an endovascular procedure, the physician punctures an artery using a needle and then navigates the vasculature to the affected area using a guidewire. The use of a catheter threaded over the guidewire delivers therapies to aid the patient, and this small incision results in minimal blood loss and a quicker recovery.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. A device for manipulating a guidewire for surgery comprising:
a housing adapted to receive a wire partially insertable into a patient at an incision site, the housing having a slot receiving a center portion of the wire between a proximal end and distal end of the wire wherein the wire is positionable to extend on a central axis of the housing; the housing supporting
a clamp assembly having a pincher and a biasing member configured to apply a biasing force on the pincher to bias the pincher to contact the wire at an at least one discrete pinch area to prevent movement of the wire with respect to the pincher.

2. The device of claim 1 wherein the housing is substantially cylindrical extending along the central axis of the housing.

3. The device of claim 2 wherein the housing has at least two opposed flattened outer surfaces.

4. The device of claim 2 wherein the housing is substantially concentric about an axis of the wire.

5. The device of claim 1 wherein the slot extends from a sidewall of the housing to the central axis.

6. The device of claim 5 wherein the slot has a height that is between 1 mm to 3 mm.

7. The device of claim 1 further comprising a groove formed on an inner wall of the slot and receiving the wire substantially on the central axis.

8. The device of claim 7 wherein the groove is V-shaped.

9. The device of claim 1 wherein the at least one discrete pinch area are discrete points on the wire.

10. The device of claim 1 wherein the pincher includes at least one outwardly extending tooth.

11. The device of claim 10 wherein the pincher includes two teeth.

12. The device of claim 1 wherein the biasing member includes at least one spring.

13. The device of claim 1 wherein the clamp assembly comprises a jaw supporting the pincher and movable against the biasing force of the biasing member.

14. The device of claim 13 wherein the jaw comprises an upper arm opposite a lower arm.

15. The device of claim 14 wherein at least one of the upper arm and lower arm support the pincher and the other of the upper arm and lower arm support the biasing member.

16. The device of claim 1 further comprising a button pressable by a human finger to move the pincher against the biasing force of the biasing member.

17. A method for manipulating a guidewire having a central portion between a proximal end and distal end of the guidewire for surgery comprising:
inserting the proximal end of the guidewire into a patient at an incision site,
inserting the central portion of the guidewire into a slot of a housing wherein the guidewire is positionable to extend on a center axis of the housing; and
clamping a clamp assembly to contact the guidewire at an at least one discrete pinch area.

18. The method of claim 17 wherein the clamp assembly applies a biasing force to contact the guidewire at the at least one discrete pinch area.

19. The method of claim 18 further comprising pressing a button to apply a force against the biasing force of the clamp assembly to release the guidewire at the at least one discrete pinch area.

20. The method of claim 17 further comprising inserting the central portion of the guidewire into a groove of the slot of the housing wherein the groove extends on the center axis of the housing.
